(19) United States
(12) Patent Application Publication
(10) Pub. No.: US 2024/0122991 A1
(43) Pub. Date: Apr. 18, 2024

(54) Transplantation of Liver Cells by Administration to the Biliary Tree of the Liver

(71) Applicant: Cambridge Enterprise Limited, Cambridge, Cambridgeshire (GB)

(72) Inventors: Fotios Sampaziotis, Cambridge, Cambridgeshire (GB); Ludovic Vallier, Cambridge, Cambridgeshire (GB); Kourosh Saeb-Parsy, Cambridge, Cambridgeshire (GB); Andrew Butler, Cambridge, Cambridgeshire (GB); Christopher Watson, Cambridge, Cambridgeshire (GB); Teik Choon See, Cambridge, Cambridgeshire (GB)

(21) Appl. No.: 18/546,746

(22) PCT Filed: Feb. 17, 2022
(86) PCT No.: PCT/EP2022/053910

§ 371 Date: Aug. 16, 2023

(30) Foreign Application Priority Data
Feb. 17, 2021 (GB) 2102235.5

(51) Int. Cl. A61K 35/407; A61K 35/413; A61P 1/16

(52) U.S. Cl. CPC A61K 35/407; A61K 35/413; A61P 1/16

(57) ABSTRACT

This invention relates to in vitro and in vivo methods for engrafting liver cells, such as cholangiocytes, into a liver comprising administering the liver cells to the biliary tree of the liver. The liver cells may be in the form of aggregates and may be administered in the direction of decreasing biliary duct diameter. This may be useful for example for the treatment of an individual with a diseased or damaged liver.

### FUNDING

The project leading to this application has received funding from the European Research Council (ERC) under the European Union's Horizon 2020 research and innovation programme (grant agreement No 741707).

### FIELD

This invention relates to methods for the administration of cells to the lumen of hollow lumen organs or branching tubular networks, for example for use in therapeutic applications, such as regenerative medicine.

### BACKGROUND

Disorders of the extrahepatic bile ducts carry considerable morbidity and mortality. Indeed, 70% of pediatric liver transplantations are performed to treat biliary atresia (Murray K. F. & Carithers R. L., Hepatology 2005, 41:1407-1432). Primary Sclerosing Cholangitis (PSC) alone accounts for 5% of US liver transplantations (Perkins J. D., Liver Transplant 2007, 13, 465-466) and biliary complications are the leading cause of graft failure following deceased liver transplantation (Skaro A. I. et al., Surgery 2009, 146:543-553; Enestvedt C. K. et al., Liver Transpl. 2013, 19:965-72). However; studies of the extrahepatic biliary epithelium have been limited by technical challenges in long-term culture and large-scale expansion of primary cholangiocytes. These challenges have so far precluded large scale experiments for drug screening and cell-based therapy targeting PSC and other cholangiopathies. Furthermore, treatment options remain limited (Gallo A. & Esquivel C. O, Pediatr. Transplant. 2013, 17:95-98; Felder S. I. et al., JAMA Surg 2013, 148:253-7-8) due to the lack of healthy donor tissue that can be used to reconstruct and replace diseased bile ducts.

In vitro expansion of liver cells, such as cholangiocytes could address this challenge and provide cells suitable for tissue engineering applications such as biliary reconstruction.

However, the administration of such cells remains problematic.

### SUMMARY

The present inventors have developed a technique that allows the direct administration and engraftment of liver cells to the liver of patients. This may be useful in regenerative medicine, for example for the treatment of disease or damaged liver tissue.

A first aspect of the invention provides a method for administering cells to a liver comprising;
administering a suspension of liver cells to the biliary tree, such that the liver cells engraft into the liver.

The method may be performed on an isolated liver for example ex vivo. Alternatively, the method may be performed in vivo. For example, the cells may be administered in a method of treatment of an individual in need thereof, for example an individual with diseased or damaged liver tissue.

A second aspect of the invention provides a liver cell suspension for use in a method of treatment of an individual in need thereof, for example an individual with diseased or damaged liver tissue, the method comprising;
administering the suspension of liver cells to the biliary tree of the individual, such that the liver cells engraft into the liver of the individual.

A third aspect of the invention provides the use of a liver cell suspension in the manufacture of a medicament for the treatment of an individual in need thereof, the treatment comprising;
administering the suspension of liver cells to the biliary tree of the liver of the individual, such that the liver cells engraft into the liver of the individual.

Preferably, the liver cells are cholangiocytes.

Preferably, the liver cells in the suspension are in the form of assemblies, clusters or aggregates comprising multiple liver cells. In some preferred embodiments, the liver cells may in the form of organoids. In other preferred embodiments, the liver cells may be in the form of cell clusters disassociated from organoids.

In methods and treatments of the first to third aspects, the liver cells may be administered in the direction of increasing bile duct diameter or more preferably in the direction of decreasing bile duct diameter.

In methods and treatments of the first to third aspects, a dimension, such as diameter, of the clusters of liver cells may correspond to the diameter of a biliary duct of the individual at a site of disease or damage.

In methods and treatments of the first to third aspects, the liver cell suspension may fill the biliary tree of the liver up to a site of disease or damage. For example, the volume of the liver cell suspension may correspond to the volume of the biliary tree from the site of administration to the site of disease or damage of the liver of the individual. In some embodiments, the volume of the suspension of liver cells may correspond to the total volume of the biliary tree of the liver of the individual.

Methods and treatments of the first to third aspects may further comprise measuring the volume of the biliary tree of the individual. In some embodiments, the volume of the biliary tree of the individual may be measured by;
administering a solution comprising a contrast agent into the biliary tree of the individual until the site of engraftment, for example a site of disease or damage, is visible by imaging, for example using a cholangiogram, and
determining the volume of the contrast agent solution administered.

The liver cells may be suspended in volume of solution that corresponds to the administered volume of the contrast agent fluid. This allows the liver cell suspension to fill the biliary tree up to the site of delivery.

Methods and treatments of the first to third aspects may further comprise washing or flushing the biliary tree of the liver before administration of the suspension to remove damaged, diseased or necrotic endogenous cells, such as cholangiocytes, from the site of engraftment and expose the underlying bile duct matrix for the administered cells to engraft.

Methods and treatments of the first to third aspects may further comprise washing or flushing the biliary tree of the liver before administration of the suspension to alter the pH of bile, for example to normalize it or return to within a physiological range. For example, the biliary tree may be flushed or washed with a buffer. The buffer may further comprises an anti-choleretic agent, such as a somatostatin analogue, to reduce the secretion of bile into the biliary tree.

Methods and treatments of the first to third aspects may further comprise retaining the liver cell suspension within the biliary tree after administration. The suspension may be retained until the liver cells engraft to the site of engraftment in the biliary tree of the liver. Preferred embodiments may comprise occluding the biliary tree after administration to reduce or prevent egress of the suspension from biliary tree. The suspension may comprise an anti-choleretic agent, such as a somatostatin, which reduces the secretion of bile into the biliary tree whilst the administered suspension is being retained.

Methods and treatments of the first to third aspects may further comprise monitoring liver function after administration of the liver cell suspension. Preferably, choleresis, concentrations of bile electrolytes, such as glucose, bicarbonate and chloride and/or bile pH are monitored. The liver cell suspension may be retained in the biliary tree of the liver until increases in liver function, for example, an increase in bile pH, electrolyte concentrations, ratios of electrolyte concentrations and/or choleresis, indicate of engraftment the liver cells.

Other aspects and embodiments of the invention are described in more detail below.

### BRIEF DESCRIPTION OF THE FIGURES

FIG. 1 shows cholangiocyte organoids (COs) rescue cholangiopathy following transplantation. (A) Experimental outline schematic. (B) Kaplan-Meier curve demonstrating animal rescue following gallbladder organoids injection; P=0.0018(**), log-rank test. (C) Magnetic Resonance Cholangiopancreatography (MRCP) demonstrating rescue of cholangiopathy following organoid injection. (D) Immunofluorescence demonstrating engraftment of Red Fluorescent Protein (RFP)-expressing gallbladder organoids in portal triads, with upregulation of intrahepatic (SOX4) markers. Scale bars; yellow, 50 μm; white, 100 μm. PV, portal vein.

FIG. 2 shows the engraftment of cholangiocyte organoids (COs) using retrograde injection in a human liver receiving Normothermic Perfusion (NMP) and improve bile properties. (A) Schematic representation of the technique for organoid injection and (B) photograph of the NMP circuit used. BD, Bile Duct; GB, Gallbladder; HA, Hepatic Artery; PV, Portal Vein; IVC, Inferior Vena Cava; L, Liver RFP, Red Fluorescent Protein; P, pump; O, oxygenator; PRC, Packed Red Cells. (C) Flow cytometry revealing absence of RFP cells in the perfusate. (D) Immunofluorescence revealing engraftment of RFP gallbladder organoids with upregulation of intrahepatic (SOX4) and loss of gallbladder (SOX17) markers. Scale bars, 50 μm. (E) Organoid injection improves bile pH and choleresis. ***P<0.001. N=3 NMP livers. Each measurement is represented by a different data point, each organ is represented by a different symbol.

FIG. 3 shows the administration of gallbladder organoids by retrograde injection in human livers receiving Normothermic 2 Perfusion (NMP). (A) Photograph of a human liver on NMP demonstrating anatomical landmarks, as well as the bile duct catheter used for administration of the Red Fluorescent Protein (RFP) expressing organoids. PV, portal vein; IVC, inferior vena cava; HA, hepatic artery; BD, Bile duct; GB, gallbladder; L, Liver. (B) Fluoroscopic images of peripheral duct cannulation. The position of the biliary catheters used for the injection of cells or carrier in the peripheral ducts of liver segments 3 and 5 respectively is shown in the top image. A cholangiogram of segment 3 following catheter placement, illustrating the peripheral position of the catheter and the area of distribution of injected the cells is shown in the bottom image. A magnified and contrast enhanced image is provided in the insert. Black arrow, sheath; red arrow, catheter tip; white arrow, cholangiogram. (C) Ultrasound imaging of the injected area of the liver revealing no duct dilation or any other abnormality at the end of the experiment. (D) Immunofluorescence analysis demonstrating engraftment, expression of key biliary markers, loss of gallbladder markers, expression of intrahepatic markers and loss of markers of other lineages in human Red Fluorescent Protein (RFP) expressing cells following transplantation in NMP human livers. Scale bars, 50 μm.

FIG. 4 shows the engraftment of gallbladder organoids in human livers receiving Normothermic Perfusion (NMP). (A) Immunofluorescence analysis demonstrating engraftment of human Red Fluorescent Protein (RFP) expressing cells following transplantation in NMP human livers. Scale bars, 100 μm. The images are complementary to FIGS. 1 and 2. (B) Quantification of gallbladder-derived RFP-expressing cells in injected vs. not injected human bile ducts; **** P<0.0001, Mann-Whitney test. The data corresponds to 3 different livers and 5 random sections per liver. Each section is represented by a data point, while each organ is represented by a different symbol.

### DETAILED DESCRIPTION

This invention relates to methods for the administration of liver cells and organoids to liver tissue, for example for the treatment of diseased or damaged liver tissue. The methods involve the administration of a suspension of liver cells in a volume that corresponds to the volume of the biliary tree of the liver of the individual.

Liver cells may include hepatocytes, macrophages, and biliary epithelial cells, such as cholangiocytes. Suitable liver cells may be primary liver cells or may be produced or expanded from primary liver cells using methods available in the art

In some preferred embodiments, the liver cells are cholangiocytes. Cholangiocytes are cells from the epithelium of biliary tissue, which is a monolayer covering the luminal surface of the biliary tree. Cholangiocytes play important roles in bile secretion and electrolyte transport in vivo. The administration of cholangiocytes as described herein may be useful for example, in the treatment of biliary disorders or diseased or damaged biliary tissue.

Suitable cholangiocytes include primary cholangiocytes, cholangiocytes produced or expanded from primary cholangiocytes using methods available in the art (see for example WO2018/234323) or cholangiocytes produced by in vitro differentiation from pluripotent cells using methods available in the art (see for example Sampaziotis et al Nat Biotech 33 (8) 845-853 (2015), WO2016/207621).

Primary liver cells, such as cholangiocytes may be obtained by any suitable method. For example, a suction catheter may be employed to obtain primary liver cells or primary liver cells may be obtained from a transbiliary forceps tissue biopsy.

In some embodiments, the liver cells in solution may be encapsulated, for example, in a hydrogel, such as alginate or Matrigel™.

The population of liver cells may be autologous i.e. the liver cells may be primary liver cells obtained from the same individual to whom they are subsequently administered or liver cells expanded from these primary liver cells (i.e. the donor and recipient individual are the same). A suitable population of liver cells for administration to a recipient individual may be produced by a method comprising providing an initial population of primary liver cells obtained from the individual and expanding the population of liver cells to produce an expanded population of liver cells for administration in suspension as described herein.

The expanded population of liver cells may be allogeneic i.e. the liver cells may be primary liver cells obtained from a different individual from the individual to whom they are subsequently administered or liver cells expanded from these primary liver cells (i.e. the donor and recipient individual are different). The donor and recipient individuals may be HLA matched and/or blood group matched to avoid rejection and other undesirable immune effects. The donor and recipient individuals may also be matched for previous viral infection history and/or viral immunity status e.g. previous CMV infection. In some embodiments, the recipient individual may receive immunosuppression therapy to reduce or prevent rejection of the allogeneic liver cells. A suitable expanded population of liver cells for administration to a recipient individual may be produced by a method comprising providing an initial population of primary liver cells obtained from a donor individual, and expanding the population of liver cells to produce an expanded population of liver cells for administration in suspension as described herein.

The liver cells in the suspension may be in the form of single cells. More preferably, the liver cells in the suspension are in the form of assemblies, clusters or aggregates comprising multiple liver cells. For example, the liver cells may in the form of organoids, or sub-organoid assemblies or clusters, for example assemblies or clusters disassociated from organoids. Suitable assemblies, clusters or aggregates of liver cells may comprise 10-500 cells, 10-100 cells, preferably 20-50 cells.

In some embodiments, the size of the aggregates of liver cells in the suspension may be dependent on the size of the biliary duct at the site of cell delivery, such as the site of damage or disease. For example, the diameter or longest dimension of the liver cell aggregates may correspond to the diameter of the biliary duct at the site of cell delivery. This may trap the aggregate in the biliary duct at the site of cell delivery and facilitate engraftment of the liver cells at the site.

Preferably, the liver cells in the suspension are organoids or cell aggregates derived therefrom. For example, cholangiocytes may be in the form of cholangiocyte organoids (COs) or cholangiocyte aggregates derived therefrom (see for example, Sampaziotis et al Nat Med 23 954-963 (2017); WO2018/234323).

Cholangiocyte organoids are three-dimensional multicellular assemblies or cysts that comprise a layer of cholangiocytes linked by tight junctions which surrounds an interior lumen and separates it from the external environment. The cholangiocytes may display polarised expression of markers, such as CFTR. Cholangiocyte organoids may display the morphology or physical characteristics of cholangiocytes. Organoids may for example comprise cilia. Tight junctions, microvilli, exosomes and/or tubular structures. The morphology and physical characteristics of organoids may be determined by standard microscopic procedures.

Cholangiocytes may express one or more biliary markers. For example, cholangiocytes may express Cytokeratin 7 (KRT7 or CK7), Cytokeratin 19 (KRT19 or CK19), Gamma Glutamyl-Transferase (GGT), Hepatocyte Nuclear Factor 1 beta (HNF1B), Secretin Receptor (SCTR), Sodium-dependent Bile Acid Transporter 1 (ASBT/SLC10A2), SRY-box 9 (SOX9) Jagged 1 (JAG1), NOTCH2, SCR, SSTR2, Apical Salt and Bile Transporter (ASBT), Aquaporin 1 and Anion Exchanger and Cystic Fibrosis Transmembrane Conductance Regulator (CFTR). Typically, at least 98% of the cholangiocytes in the population may co-express CK7 and CK19 following 20 passages in the expansion medium as described herein.

The liver cells in the suspension, whether in the form of aggregates, organoids or individual cells, may be free or substantially free from other cell types i.e. the population of liver cells may be homogeneous or substantially homogeneous. For example, the population of cells in the suspension may contain, 80% or more, 90% or more, 95% or more, 98% or more or 99% or more liver cells.

In some preferred embodiments, a method described herein may comprise washing or flushing the biliary tree of the liver before administration of the liver cell suspension. This may be useful for example, in dislodging or removing diseased or damaged liver cells at the engraftment site or modifying the microenvironment at the engraftment site to support cell survival, for example through pH optimisation.

The biliary tree may be flushed or washed with a wash solution. The wash solution may comprise a buffer, such as PBS or HEPES. This may be useful for example in neutralising the pH in the biliary ducts of the biliary tree.

The biliary tree may be flushed or washed until there is no reduction in the number of returning cells in the effluent in 2 consecutive washes. For example, the biliary tree may be flushed or washed 1 or more, 2 or more, 3 or more, 4 or more or 5 or more times with fresh wash buffer.

In some embodiments, the wash solution may further comprise an anti-choleretic agent, e.g. Somatostatin (STT), Somatostatin analogues, CFTR-inh172, or octreotide. This may be useful for example in reducing bile secretion in the biliary tree.

In other embodiments, an anti-choleretic agent may be administered systemically to the liver and/or the individual.

The liver cell suspension may fill the biliary tree of the liver up to the site at which cell engraftment is required, for example, a site of disease or damage. For example, the volume of the liver cell suspension may correspond to the volume of the biliary tree up to the engraftment site in the biliary tree of the individual. This exposes the bile ducts in the biliary tree up to and including the desired site of engraftment to the cell suspension without damaging the bile ducts by subjecting them to excess pressure.

In some embodiments, a site of disease or damage in the biliary tree suitable for engraftment as described herein may be identified by imaging techniques, such as trans biliary intravascular ultrasound and MR-cholangiography.

In some embodiments, the liver cell suspension may completely fill the biliary tree (i.e. all the bile ducts, including terminal branches may be exposed to the suspension). For example, the volume of the suspension may correspond to the volume of the intrahepatic biliary tree or the entire biliary tree of the liver of the individual. This exposes all of the bile ducts in the biliary tree to the cell suspension without damaging the bile ducts by subjecting them to excess pressure.

The volume of the biliary tree varies between individuals. In preferred embodiments, a method described herein may comprise measuring the volume of the biliary tree of the individual up to the engraftment site before administration of the cell suspension. This allows the volume of the cell suspension to be adjusted to the specific volume required to deliver liver cells to the engraftment site in that individual.

In some embodiments, the volume of the biliary tree of the individual may be measured by radiological techniques, including magnetic resonance (MR) imaging, for example MR-cholangiography.

In other embodiments, the volume of the biliary tree of the individual may be measured by;
administering a solution comprising a contrast agent into the biliary tree of the individual until the desired engraftment site in said tree is visible by imaging, and
determining the injected volume of the contrast agent solution.

Suitable contrast agents are well known in the art and include gadolinium agents, and water-soluble iodine-based agents. Suitable techniques for the injection and imaging of contrast agent solutions are well known in the art (see for example Gastrointestinal Endoscopy 62 4 2005 480-484).

Suitable techniques for imaging contrast agents are well known in the art and include cholangiography, endoscopic retrograde cholangiopancreatography (ERCP), and percutaneous transhepatic cholangiography (PTC).

Liver cells may be resuspended in a volume that corresponds to the measured volume of the contrast agent solution that was injected into the individual. The liver cells may be suspended in a cell culture medium. Suitable basal cell culture media are well known in the art.

The cell culture medium may further comprise a Rho kinase (ROCK) inhibitor. The ROCK inhibitor may be useful in priming the liver cells and increasing their attachment to the site of delivery in the biliary tree. Suitable ROCK inhibitors commercially available and include Y-27632 and fasudil.

The cell culture medium may further comprise a buffer.

The cell culture medium may further comprise one or more components that prevent cell attachment to solid surfaces, such as catheter walls. Suitable components are well known in the art and include BSA.

The biliary tree is an assembly of bile ducts within the liver that collect bile from the hepatic parenchyma, so that it can be transported to the duodenum.

In some embodiments, the suspension of liver cells is administered to the biliary tree of the liver of the individual in the direction of increasing bile duct diameter i.e. in the direction of bile flow. This direction may be referred to as the “antegrade direction” herein. This may be useful, for example for percutaneous administration for segmental delivery of cells.

In more preferred embodiments, the suspension of liver cells is administered to the biliary tree in the direction of decreasing bile duct diameter i.e. towards the terminal branches of the biliary tree. This direction may be referred to as the “retrograde direction” herein (i.e. opposite to the direction of bile flow). The cell suspension may be administered to the biliary tree of the liver of the individual in a retrograde direction.

The suspension may be administered into the common hepatic duct, the left or right hepatic duct, or one of the ducts of the biliary tree. Preferably, the suspension is administered to the biliary tree by injection, for example using a cannula or catheter.

In some embodiments, the suspension may be administered through a catheter inserted into the common, left or right hepatic duct; or a catheter introduced using an endoscopic route or percutaneously, for example percutaneously into the gallbladder and then into the biliary tree. In some embodiments, the suspension may be administered via a contralateral percutaneous administration. For example, a right sided bile duct may be punctured to position a catheter as peripheral as possible, or wedged in the left ducts of the biliary tree. A suitable cannula or catheter may be designed or shaped to wedge against an intrahepatic tributary of the bile duct. A suitable cannula or catheter may be internally coated with a non-adhesive reagent, for example a coating protein, such as BSA, to prevent cell attachment to the internal surfaces of the cannula or catheter. In some embodiments, the interior surface of the cannula or catheter may be exposed to a coating protein solution, such as a BSA solution, FBS or protein rich medium, such that the coating protein adheres to the interior surface and blocks the attachment of cells.

The cells are preferably injected under conditions that do not damage the cells. For example, the cells may be injected at a hydrodynamic pressure that is insufficient to cause damage to the cells.

Preferably, surfaces in contact with the cell suspension, such as the inner surfaces of the cannula or catheter are coated to prevent adhesion of the cells in the suspension. For example, surfaces may be coated with a protein, such as bovine serum albumin.

The liver cells may be delivered to a site of engraftment in the biliary tree. The site of engraftment may be a site of damage or disease. The location of the site of engraftment may depend on the disease. For example, the site of engraftment in the biliary tree of an individual with primary bile cholangitis (PBC) may be the terminal ductules, and the site of engraftment in the biliary tree of an individual with primary sclerosing cholangitis (PSC) may be the intermediate ducts.

Preferably, the cannula or catheter used to administer the cell suspension to the biliary tree is kept in place until the liver cells have engrafted to the liver tissue.

Following administration, the liver cell suspension may be held or retained within the biliary tree of the liver until the liver cells engraft to the liver. For example, one or more bile ducts of the biliary tree may be reversibly occluded following injection to hold the suspension in place, reduce or prevent egress of the suspension out of the biliary tree. A bile duct may be occluded by physically blocking the lumen of the bile duct without causing pressure damage or necrosis. This prevents back-flow of the cell suspension. For example, the common hepatic duct, left or right hepatic duct, or (one or more) smaller bile ducts may be occluded, depending on the site into which the cell suspension is administered. Suitable methods of occluding bile ducts are well known in the art and include balloons, tubes, stents and beads.

In some preferred embodiments, the biliary tree may occluded by inflating a balloon in the bile duct following administration of the cell suspension. Balloon catheters that can be used for this purpose are commercially available.

The biliary tree may be occluded until the liver cells have engrafted with the liver tissue For example, the biliary tree may be occluded for 2 or more hours, 3 or more hours or 4 or more hours. After engraftment, the occlusion may be removed or reversed.

The biliary tree of the liver may be monitored following administration of the cell suspension to determine when liver cells have engrafted with the liver tissue. For example, one or more parameters of liver function may be monitored or measured periodically. Changes in the one or more parameters may be indicative of the engraftment of the liver cells and the improved functioning of the liver tissue.

Preferably, the one or more parameters include bile pH, glucose/bicarbonate levels and/or choleresis (volume of the bile produced since the administration of cells). An increase in bile pH and/or bile volume may be indicative of the engraftment of cholangiocytes and the improved functioning of biliary tissue in the liver.

Bile pH may be determined using standard biochemical assays. Bile volume may be determined, for example, by free drainage from a catheter over a set amount of time.

The methods described herein may be useful in administering liver cells ex vivo. For example, the liver to which the cells are administered may be an isolated liver on a perfusion machine. Following engraftment, the liver may be transplanted into an individual. The liver may be a whole liver or a liver lobe or segment.

The methods described herein may be useful in administering liver cells in vivo. For example, the liver may be in situ in an individual. This may be useful for example in a method of treatment of an individual, for example an individual with diseased or damaged liver tissue. Suitable individuals include mammals, preferably humans.

In some embodiments, the individual may have diseased or damaged biliary tissue, for example diseased or damaged bile duct epithelial tissue. The individual may for example have a biliary disorder. Administration of cholangiocytes as described herein may be useful in the treatment of a biliary disorder.

A biliary disorder is a condition in which the biliary tissue in an individual is damaged, defective or otherwise dysfunctional, for example, disorders characterised by damage to or destruction of bile ducts, aberrant bile ducts or the absence of bile ducts. Biliary disorders may include biliary tissue injury, ischaemic strictures, traumatic bile duct injury and cholangiopathies, for example inherited, developmental, autoimmune and environment-induced cholangiopathies, such as Cystic Fibrosis associated cholangiopathy, drug induced cholangiopathy, Alagille Syndrome, polycystic liver disease, primary biliary cirrhosis (PBC), primary sclerosing cholangitis (PSC), AIDS associated cholangiopathy, disappearing bile duct syndrome, biliary cancer, ductopenias such as adult idiopathic ductopenia, post-operative biliary complications, and biliary atresia.

In some preferred embodiments, administration of a population of cholangiocytes in suspension as described herein may be useful in the treatment of ductopenias, for example ischaemic ductopenia, congenital ductopenia, such as alagille syndrome, metabolic ductopenia, complex diseases, such as intrahepatic PSC and PBC, drug induced ductopenia, vanishing bile duct syndrome and conditions affecting the biliary tree.

Administration of a composition in accordance with the present invention is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc., is within the responsibility of general practitioners and other medical doctors.

A composition comprising liver cells may be administered alone or in combination with other treatments, either simultaneously or sequentially dependent upon the condition to be treated. For example, other therapeutic agents or cells may be administered via the bile ducts, or systemically, for example via the portal vein (PV), hepatic vein (HV) or hepatic artery (HA).

Other aspects and embodiments of the invention provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope of the present invention.

All documents and sequence database entries mentioned in this specification are incorporated herein by reference in their entirety for all purposes.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

### EXPERIMENTAL

Materials and Methods

Ex Vivo Normothermic Perfusion of Donor Livers

The metra (OrganOx, Oxford, UK) normothermic liver perfusion device was used for ex vivo perfusion of human livers as previously described (Haghverdi et al Nat. Methods. 13, 845-848 (2016)). The machine, which is clinically used for preservation of livers for transplantation (A. Lanzini, BILE. Encycl. Food Sci. Nutr. 471-478 (2003)) enables prolonged automated organ preservation by perfusing it with ABO-blood group-compatible normothermic oxygenated blood. The perfusion device incorporates online blood gas measurement, as well as software-controlled algorithms to maintain pH, P02 and PC02 (within physiological limits), temperature and mean arterial pressure within physiological normal limits. In brief, the hepatic artery, portal vein, inferior vena cava and bile duct were cannulated, connected to the device and perfusion commenced.

Bile Duct Cannulation

Cannulation of the bile duct was achieved by inserting two Fr sheaths into the common bile duct under fluoroscopy guidance, followed by cannulation of the left and right hepatic ducts and subsequently segment 3 and segment ducts respectively, using two 2.7 Fr microcatheters via the sheaths. Peripheral placement of the microcatheters was confirmed by cholangiogram with small amount of ionic contrast medium. Cells were injected into segment 3 and carrier was injected into segment 5.

Cell Delivery

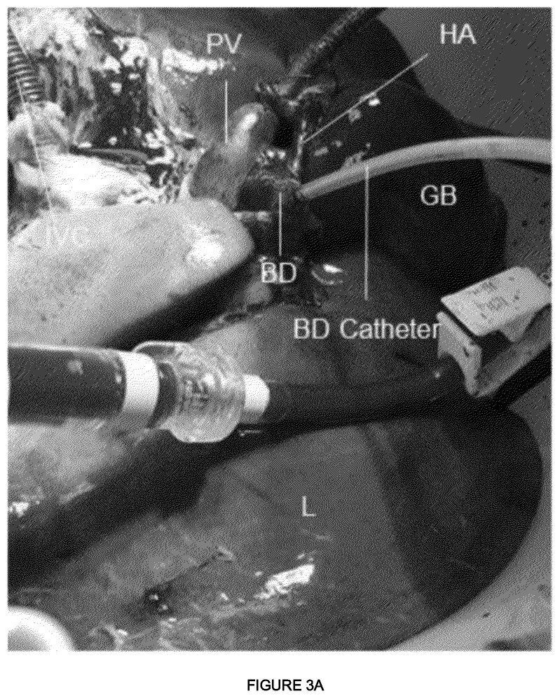

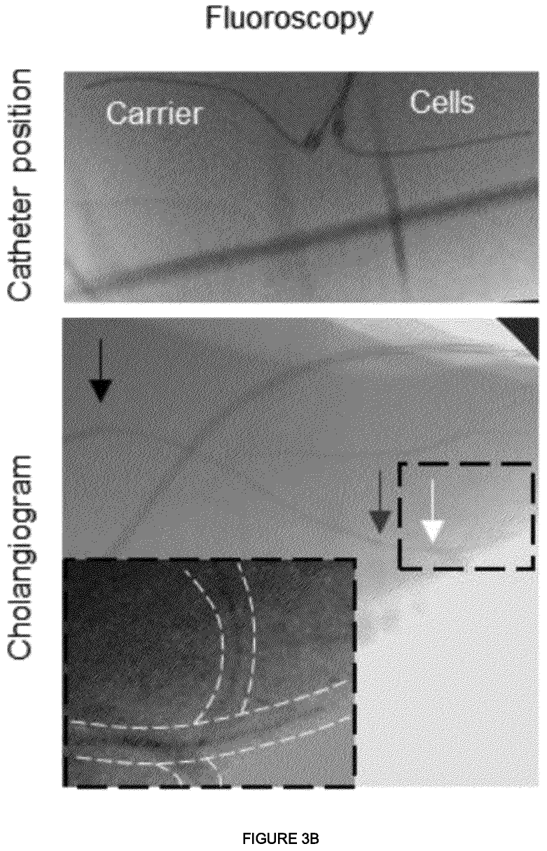

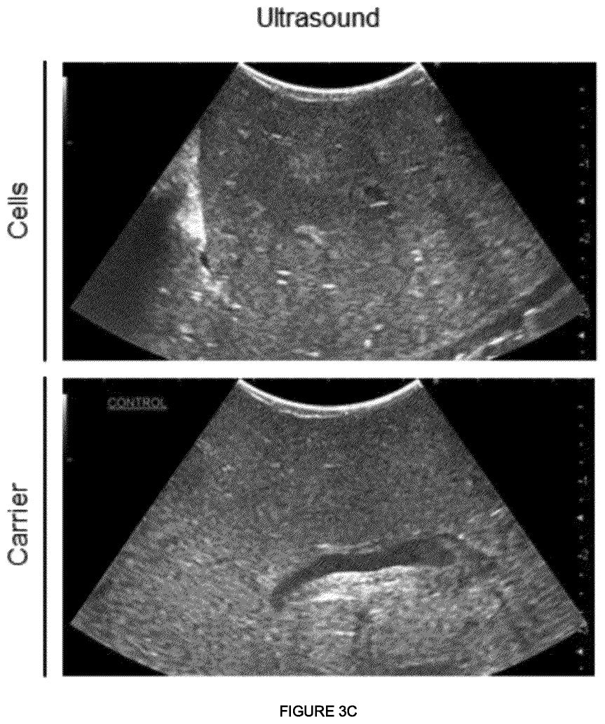

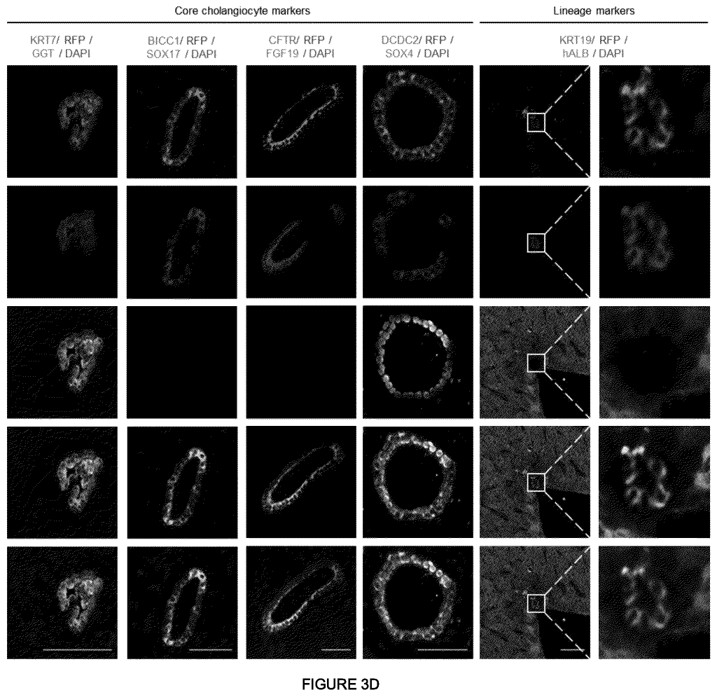

RFP-expressing organoids were mechanically dissociated to a mixture of small clumps and single cells and approximately 10×106 RFP-expressing cells were administered in a peripheral duct of segment 3 with a distribution area of ˜2 cm3, which was cannulated under fluoroscopic guidance to maximize cell delivery (see Bile duct cannulation section) (FIG. 3B). Carrier medium was delivered in a peripheral branch of segment using the same technique and the organ was maintained on NMP for up to 100 hours.

Quantification of Transplanted Cells in Human Livers

3 human livers injected with RFP-labelled gallbladder organoids were analysed. Sections were obtained from the area of the distribution of the cells (˜2 cm3). 5 sections per liver and a total of 4,463 cells were analysed.

Bile Aspiration

Bile duct cannulation was performed as described in the relevant section. Following cannulation, microfluidic catheters (CMA Microdialysis Catheter, Harvard Bioscience Inc, USA) were placed into the respective segmental ducts using a guide wire exchange technique. The inner and outer shaft of the catheter and the inlet and outlet tubing are made of polyurethane and the membrane composed of polyarylethersulphone with a membrane pore size of 100 kDa and outer diameter of 0.4 mm. The inlet tubing for each catheter was connected to a portable battery driven CMA 107 Microdialysis Pump (Harvard Bioscience Inc, USA) and the pump was set to aspirate at a rate of 1 μl/min.

Bile Volume and pH Measurements

Measurements were performed in n=3 different livers. A minimum of 2 repeat measurements were performed for each liver increasing to where possible, as previously described (Haghverdi et al Nat. 12 Biotechnol. 36, 421-427 (2018).). Bile volume was normalised over the volume of the bile ducts producing it, which corresponds to the volume of distribution of the cells or the carrier in the control arm. This was calculated using the volume of the contrast medium required to delineate these ducts on cholangiogram. Please note all catheters were primed prior to volume measurements.

Ultrasound Imaging

The liver was imaged ex-vivo in a normothermic perfusion device using a Hitachi Aloka Arrieta V70 and a 10 Mhz hand-held probe. Images were obtained in axial and sagittal planes and assessment of the portal vein, hepatic veins and their major branches was carried out. The intrahepatic bile ducts were also assessed, with particular attention to segment 3 where the organoids had been instilled, and a control area in segment 5 receiving carrier.

Results

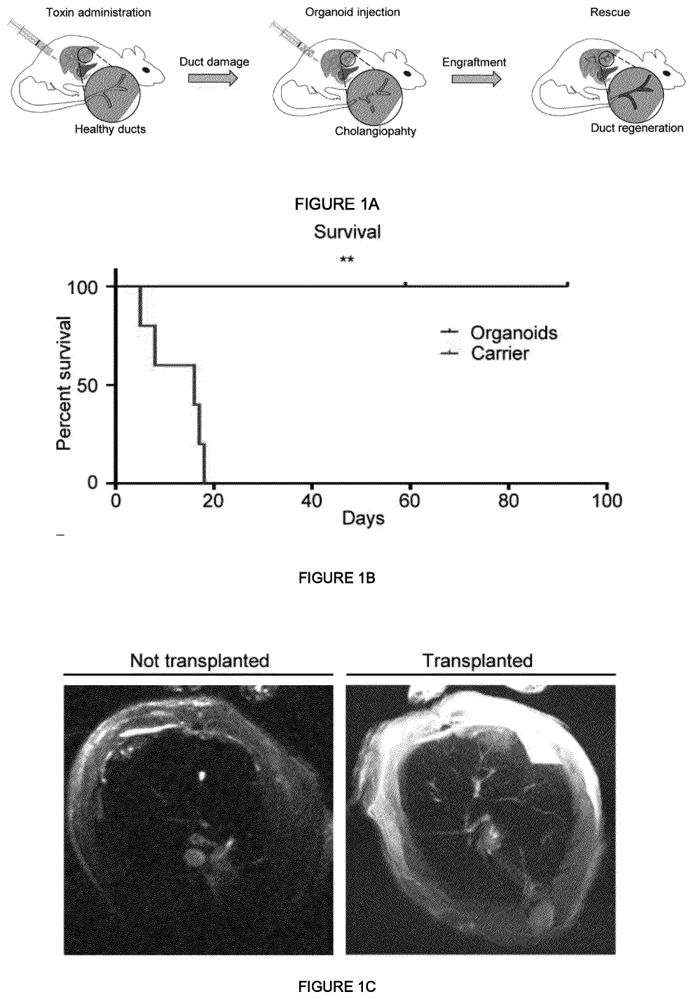

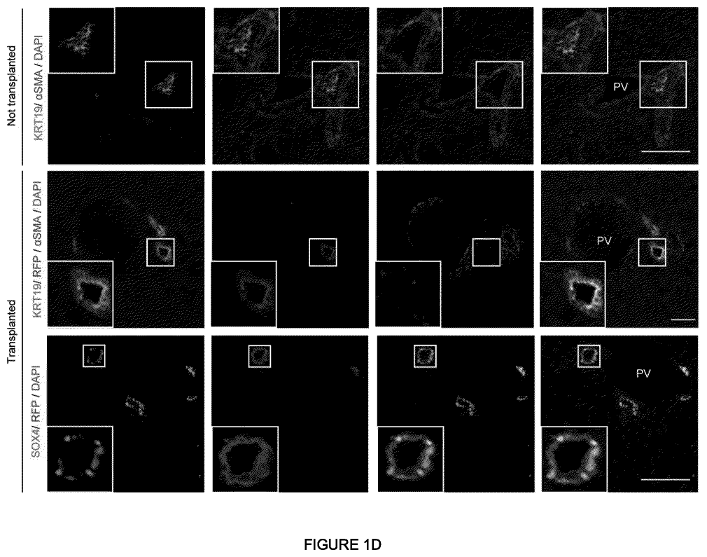

We induced cholangiopathy in immunodeficient mice using 4,4′-methylenedianiline (MDA) (Lee et al Nat Protoc 14 1884-1925 (2019)) (FIG. 1A-B) and attempted to rescue the phenotype with intraductal delivery (Bernsten et al Am. J. Physiol.—Gastrointest. Liver Physiol. 314, G349-G359 (2018)) of human gallbladder organoids expressing Red Fluorescen Protein-expressing (RFP). Control animals receiving carrier medium without cells lost weight and died within 3 weeks (FIG. 1B), developing cholestasis and cholangiopathy demonstrated by IF), histology and Magnetic Resonance Cholangio-Pancreatography (MRCP) (FIG. 1C). On the contrary, animals receiving organoids were electively culled at the end of the experiment and survived for up to 3 months with resolution of cholangiopathy and normal serum biochemistry (FIG. 1B-C). The transplanted gallbladder cholangiocytes engrafted in various size intrahepatic ducts (FIG. 1D) corresponding to ˜25-55% of the regenerated biliary epithelium, and assumed an intrahepatic identity by losing gallbladder (SOX17) and expressing intrahepatic markers (SOX4, DCDC2, BICC1) (FIG. 1D). Core biliary markers (KRT7, KRT19, CFTR) were also expressed, while we observed YAP activation both in engrafted and native cells in accordance with previous reports (Pepe-Mooney et al Cell Stem Cell. 25, 23-38.e8 (2019)). Of note, we never observed expression of other hepatic lineage markers such as albumin indicating that cholangiocyte organoid plasticity is likely to be limited to their biliary lineage. Furthermore, the engrafted cells expressed proliferation markers at similar levels to native mouse cholangiocytes; while abnormal growth or tumour formation was never noticed in all the analyses performed (FIG. 1C, 1 D), including T1 weighed body MR imaging at the end of the experiment. Thus, organoid transplantation provides the healthy cells required to repair the damaged epithelium and rescue acute injury.

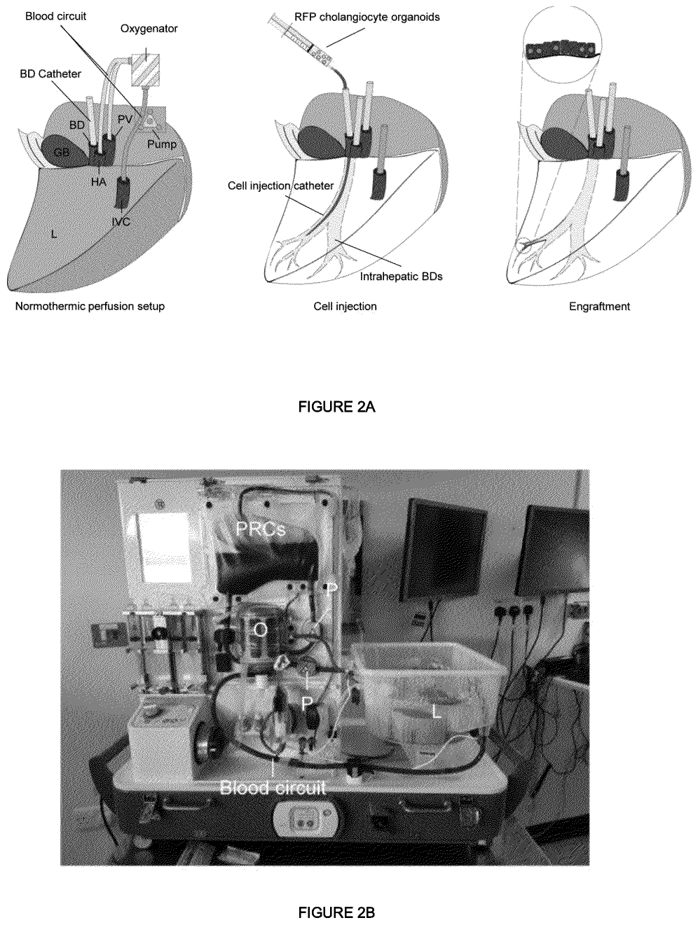

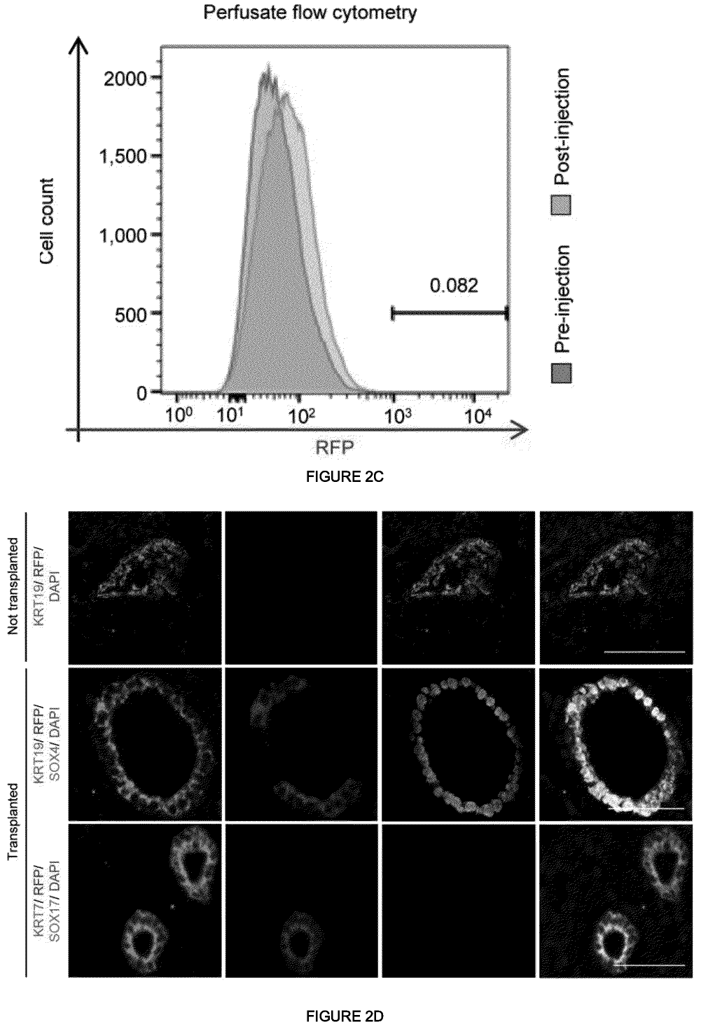

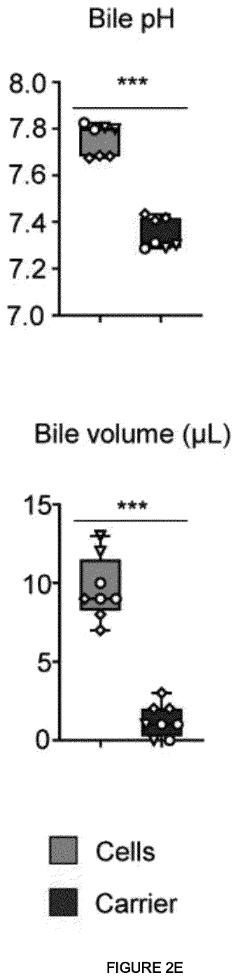

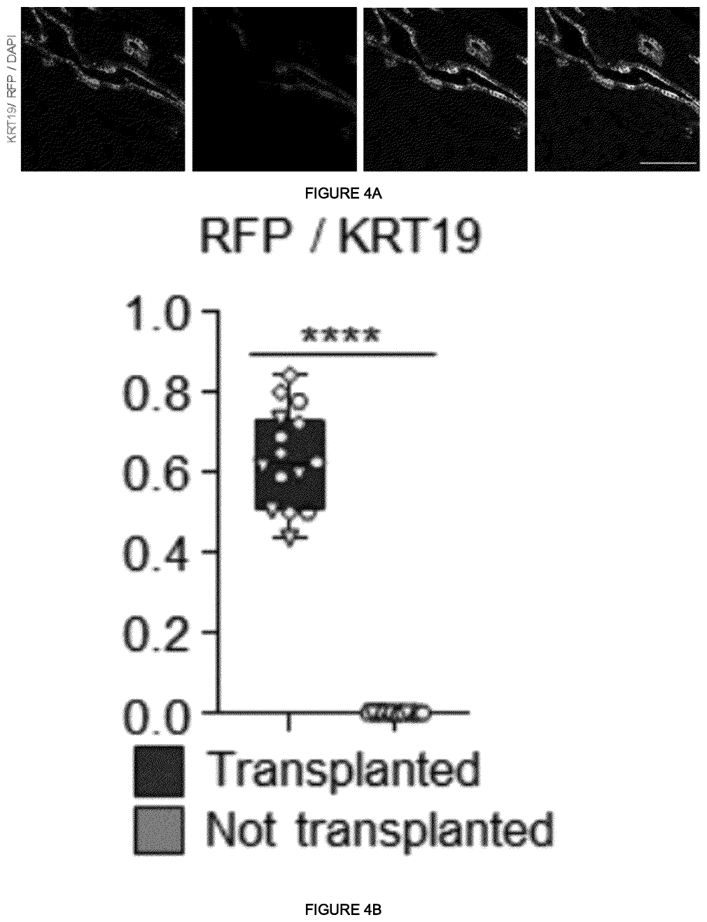

To assess the therapeutic potential of our cells for repairing human bile ducts, RFP gallbladder organoids were injected in the intrahepatic ducts of deceased transplant donor livers (n=3) with a bile pH<7.5 at the start of the experiment, signifying ischaemic duct injury. The organs were perfused with oxygenated blood and nutrients at normal body temperature (Nasralla Nature. 557, 50-56 (2018)); FIG. 2A-1B, 3A) for up to 100 hours in order to maintain a near-physiological microenvironment. Importantly, the organoids were delivered in a terminal branch of the intrahepatic ducts under fluoroscopic guidance to minimize the area of distribution of the cells and maximize cell density (FIG. 3B). At the end of the experiment, ultrasound imaging revealed no evidence of duct dilatation or obstruction (FIG. 3C), while RFP-expressing cells were not detected in the perfusate by flow cytometry, confirming that the injected cells remained in the biliary compartment (FIG. 2C). More importantly, the transplanted organoids engrafted in the intrahepatic biliary tree (FIG. 2D, 4A), with RFP cells regenerating ˜40-85% of the injected ducts (FIG. 4B); and expressing key biliary markers (KRT7, KRT19, CFTR, GGT). Furthermore, engrafted gallbladder organoids exhibited loss of gallbladder (SOX17) and upregulation of intrahepatic (SOX4, BICC1, DCDC2) markers without differentiation to other hepatic lineages (FIG. 2D, 3D, 4A-3B). Thus, at the end of the experiment, the injected ducts consisted of a mixture of native and transplanted cholangiocytes (FIG. 4A-3B), with multiple transition points between donor and recipient cells and no evidence of cholangiopathy (FIG. 2D, 3D, 4A). Conversely, control ducts not receiving cells demonstrated evidence of ischaemic injury with loss of epithelial continuity and sloughing of cells in the duct lumen (FIG. 2D). We subsequently characterised the impact of engraftment on organ function. Physiologically, cholangiocytes modify the composition and pH of bile through water transfer and bicarbonate secretion (Yoo et al Gut Liver. 10, 851-8 (2016)). Therefore, we compared the bile from organoid-injected vs. carrier-injected ducts. Accordingly, bile aspirated from ducts injected with cells exhibited higher pH and volume (FIG. 2E) confirming that transplanted cholangiocytes retain their function to modify bile composition.

1. A method for the administration of cells to a liver comprising;
administering a suspension of liver cells to the biliary tree of the liver in the direction of decreasing biliary duct diameter, such that the liver cells engraft into the liver.

2. A method according to claim 1 wherein the method is an in vitro or ex vivo or in vivo method.

3. A method of treatment of an individual with a diseased or damaged liver comprising;
administering a suspension of liver cells to the biliary tree in the direction of decreasing biliary duct diameter, such that the liver cells engraft into the liver.

4. A suspension of liver cells for use in a method of treatment of an individual with a diseased or damaged liver comprising;
administering a suspension of liver cells to the biliary tree of the liver in the direction of decreasing biliary duct diameter, such that the liver cells engraft into the liver.

5. Use of a suspension of liver cells in the manufacture of a medicament for use in a method of treatment of an individual with a diseased or damaged liver comprising;
administering a suspension of liver cells to the biliary tree of the liver in the direction of decreasing biliary duct diameter, such that the liver cells engraft into the liver.

6. A method according to any one of claims 1 to 3, a suspension for use according to claim 4 or use of claim 5 wherein the liver cells in the suspension are in the form of aggregates.

7. A method according to any one of claims 1 to 3 or 6, a suspension for use according to claim 4 or 6 or use of claim 5 or 6 wherein the liver cells are suspended in a culture medium comprising a buffer and a Rho kinase inhibitor.

8. A method according to claim 7, a suspension for use according to claim 7 or use according to claim 7 wherein the culture medium further comprises an anti-choleretic agent and/or a non-adhesive reagent.

9. A method according to any one of claims 1 to 3, or 6 to 8, a suspension for use according to claim 4, or 6 to 8 or use of any one of claims 5 to 8 wherein the liver cells are cholangiocytes.

10. A method according to any one of claims 1 to 3, or 6 to 9, a suspension for use according to any one of claim 4 or 6 to 9 or use of any one of claims 5 to 9 wherein the method comprises measuring the volume of the biliary tree of the individual up to a desired site of engraftment and suspending the liver cells in a volume corresponding to the volume measured for the individual.

11. A method according to claim 10, a suspension for use according to claim 10 or use according to claim 10, wherein the volume of the biliary tree of the individual is measured by;
administering a fluid comprising a contrast agent into the biliary tree of the individual until the desired site of engraftment is visible in on imaging, and
determining the injected volume of the contrast agent fluid.

12. A method according to claim 10, a suspension for use according to claim 10 or use according to claim 10, wherein the volume of the biliary tree of the individual is measured by MR imaging

13. A method according to any one of claims 1 to 3, or 6 to 12, a suspension for use according to any one of claim 4 or 6 to 12 or use of any one of claims 5 to 12 wherein the method comprises flushing the biliary tree of the liver with a wash solution comprising a buffer before administration of the suspension of liver cells.

14. A method according to claim 13, a suspension for use according to claim 13 or use according to claim 13 wherein the pH of the biliary tree following said flushing is non-toxic to the administered liver cells.

15. A method according to claim 13 or 14, a suspension for use according to claim 13 or 14 or use according to claim 13 or 14 wherein said flushing removes diseased or damaged cells at the site of engraftment.

16. A method according to any one of claims 1 to 3, or 6 to 15, a suspension for use according to any one of claim 4 or 6 to 15 or use of any one of claims 5 to 15 further comprising administering to the individual an anti-choleretic agent to reduce bile secretion.

17. A method according to any one of claims 1 to 3, or 6 to 16, a suspension for use according to any one of claim 4 or 6 to 16 or use of any one of claims 5 to 16 wherein the cell suspension is injected into the biliary tree through a catheter or cannula.

18. A method according to claim 14, a suspension for use according to claim 14 or use according to claim 14 wherein the interior surface of the catheter or cannula is coated with protein to prevent cell adhesion.

19. A method according to any one of claims 1 to 3, or 6 to 18, a suspension for use according to any one of claim 4 or 6 to 18 or use according to any one of claims 5 to 18 wherein the method comprises retaining the suspension of liver cells within the biliary tree following said administration.

20. A method according to claim 19, a suspension for use according to claim 19 or use according to claim 19 wherein the suspension of liver cells is retained in the biliary tree by occluding the lumen of a bile duct of said tree to reduce or prevent egress of the suspension.

21. A method according to claim 20, a suspension for use according to claim 20 or use according to claim 20 wherein the biliary tree is occluded by inflating a balloon in said duct.

22. A method according to claim 20, a suspension for use according to claim 20 or use according to claim 20 wherein the biliary tree is occluded by biodegradable beads.

23. A method according to any one of claims to 22, a suspension for use according to any one of claims to 22 or use according to any one of claims to 22 wherein the biliary tree is occluded for 30 minutes or longer.

24. A method according to any one of claims 1 to 3, or 6 to 23, a suspension for use according to any one of claim 4 or 6 to 23 or use according to any one of claims 5 to 23 wherein the method comprises monitoring the pH, electrolyte content and/or volume of the bile produced in said liver following said administration, wherein an increase in pH, electrolyte content and/or volume of the bile is indicative that the liver cells are engrafted.
